(19) United States
(12) Patent Application Publication
(10) Pub. No.: US 2017/0340512 A1
(43) Pub. Date: Nov. 30, 2017

(54) ENERGIZING BED

(71) Applicant: Steven-Hunt Kang, Tainan (TW)

(72) Inventor: Steven-Hunt Kang, Tainan (TW)

(21) Appl. No.: 15/163,723

(22) Filed: May 25, 2016

(51) Int. Cl. A61H 33/14; A61L 2/10; A61M 16/10; A61F 7/00

(52) U.S. Cl. CPC A61H 33/14; A61F 7/0053; A61L 2/10; A61M 16/1005; A61H 2033/141; A61H 2201/10; A61H 2201/50; A61H 2033/143

(57) ABSTRACT

A energizing bed, primarily comprising an enclosed space of the bed with a power control box and a door for entering and exiting. A heating apparatus is connected to the base board of the bed while an anion generator and oxygen generator are attached to the outside of the bed body. One of the bed ends has air holes for supplying anions and oxygen as well as being the side to mount the power control box, which will control the heating apparatus, anion generator, and oxygen generator. This allows the person to be treated to lie down on the bed and revitalize oneself by allowing cell metabolism to happen in its ideal environment.

### BACKGROUND OF THE INVENTION

### 1. Field of the Invention

This invention relates to an energizing bed, particularly to one can revitalize cells while lying down in an enclosed environment with heat, anions and oxygen.

### 2. Description of the Prior Art

As the old saying goes, you gotta move if you wanna live. The movement creates rise in body temperature.

Exercise of any form isn't just to build muscle, but to raise your body temperature. We sweat because our body temperature is raised. Sweating also is a detoxification process in of itself, which most people know. What people do not know is that raising body temperature through exercise is to provide cells with the proper environment of maintaining healthy state, as cells at the current body temperature of 36.5 degrees Celsius are only half-awake while metabolism is cycling cells at 105 cells per second. Cells that divide in this lower temperature retain their current status as healthy, not healthy, or even cancerous rather than splitting to become brand new healthy cells. Thus when we get a cold or sick, the body temperature is raised and our cells start replacing the dying sick cells with new healthy ones, naturally bringing our health back to healthy state. Cells require oxygen as nourishment. However, exercise rooms typically do not have enough oxygen needed and thus fall short of the requirements for the cells even if the temperature is right.

The forest is a good source of oxygen, and instead of exercising one would take a stroll and see the sights, thus taking in oxygen, but you do not have the temperature the cells need to properly make new cells.

Another reason for cell mutation: pH changes with eating meat. A normal human body's acidity is typically between 7.35 to 7.45 pH. As the body automatically adjusts to our eating, excessive meat leads the body's pH levels to drop in order to accommodate balancing the acidity of the meat. However when the body's pH levels drop to 7.15 to 7.35 pH, our body's ability to process oxygen drops by 69.5 percent, in effect only allowing us to use about 6% of the air's oxygen. The lack of oxygen is often cited as a cause for illnesses such as cancer, thus whether you are absorbing enough oxygen also becomes an important factor.

There are also free radicals created from cell metabolism process, white blood cells attacking foreign objects and such. Anions can help neutralize free radicals and is the key to helping the body to become healthy when free radicals are eliminated.

### SUMMARY OF THE INVENTION

The objective of this invention is to provide an enclosed space of bed with a heated of base board and provide the enclosed space with anions and oxygen to facilitate cell revitalization to promote healthier oneself.

The energizing bed in the present invention includes an enclosed space for a bed, with two air holes, a heating apparatus connected to the bed bottom to provide heat, an anion generator and an oxygen supplier are placed outside the unit with the pipe feeding anion and oxygen in, a power control box to supply and regulate the power used by the machine and its components. Everything together makes for a proper environment where cells can revitalize.

### BRIEF DESCRIPTION OF DRAWINGS

This invention will be better understood by referring to the accompanying drawings, wherein:

FIG. 1 is a perspective view of a preferred embodiment of an energizing bed in the present invention;

FIG. 2 is a perspective view of the preferred embodiment of an energizing bed in the present invention; and

FIG. 3 is a cross-sectional view of the energizing bed in using condition in the present invention.

### DETAILED DESCRIPTION OF THE PREFERRED EMBODIMENT

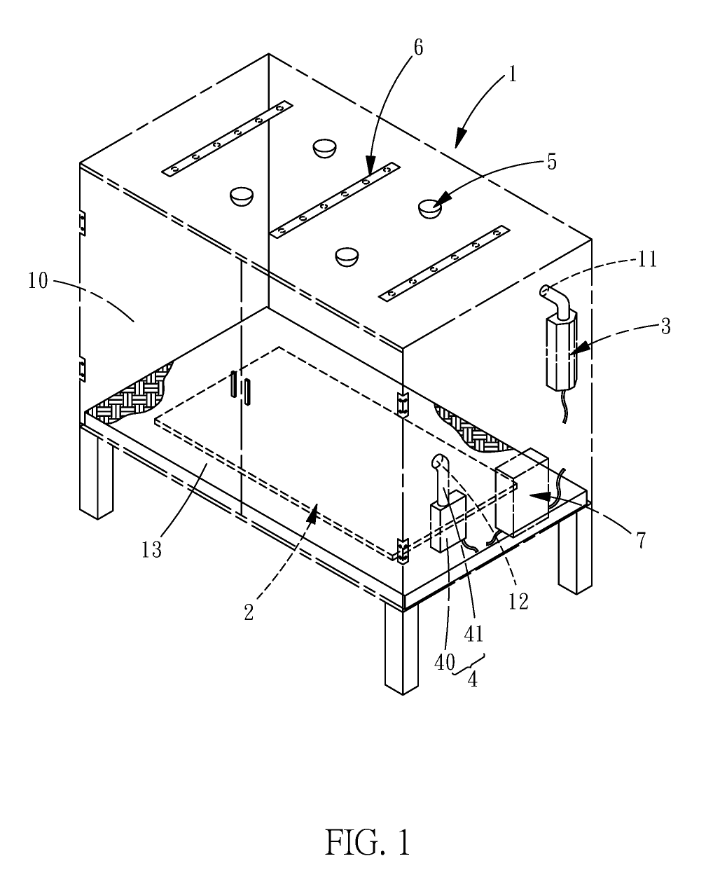

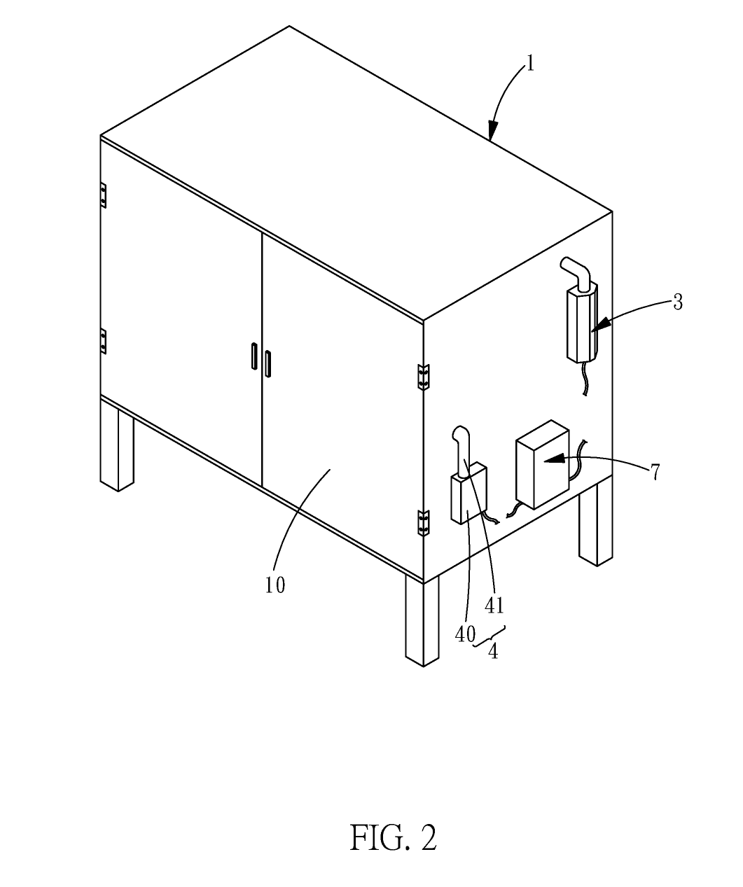

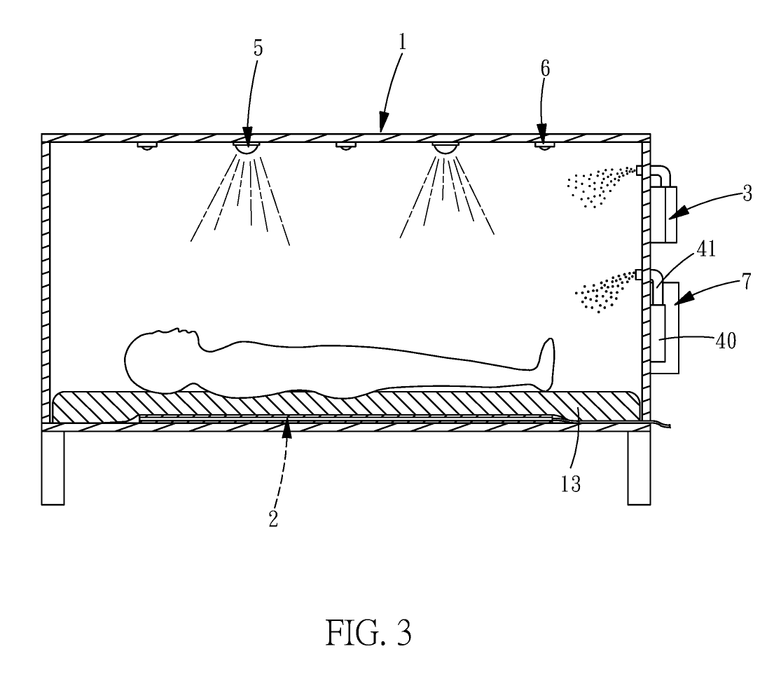

As shown in FIGS. 1 to 3, a preferred embodiment of an energizing bed in the present invention includes a bed 1, a heating apparatus 2, an anion distributor 3, an oxygen supplier 4, heat lamp 5, an ultraviolet sanitation light 6 and a power control box 7 as main components combined together.

An enclosed space for a bed 1, the bed 1 one side has outward-opening doors 10, and on the bed 1 there are air holes 11, 12 and a base board 13.

The heating apparatus 2 is set on the bed 1 to be below the base board 13, allowing for the heating apparatus 2 to send hot water at specified temperatures through the base board 13, allowing for the base board 13 to have a specified temperature through heating apparatus 2.

The anion distributor 3 is mounted at the outside of the bed 1, with piping through air hole 11 to deliver the anions to the inside of enclosed space.

The oxygen supplier 4 is mounted on the outside of the bed 1, composed of the oxygen generator 40 and its feed Pipe 41, with the feed pipe 41 going through air hole 12 to deliver oxygen to the inside of enclosed space.

The heat lamp 5 is installed top of the bed 1, mainly used to help bed 1 temperature to its needed levels, and the heat lamp 5 also can be the heating fan.

The ultraviolet sanitation light 6 is installed inside the enclosed space of the bed 1 to keep the internal space clean.

The power control box 7 is located on the outside the bed 1, and controls the power for the heating apparatus 2, the anion distributor 3, the oxygen supplier 4, the heat lamp 5 and the ultraviolet sanitation light 6.

In using, the bed 1 through the doors 10 and close it behind them, creating a sealed space. Then start the power control box 7, which powers the heating apparatus 2, the anion distributor 3, and oxygen supplier 4. At this point the base board 13 will have heated up the person to be treated body temperature, which, when combined with the sufficient anions and oxygen inside of the bed 1, will facilitate the ideal environment for cell metabolism.

The invention has the following advantages as can be seen from the foresaid description.

1. The bed 1 of this invention is an enclosed space, so as to prevent heat leakage or oxygen concentration drops.

2. The heating apparatus 2 of this invention is using a method similar to northern ground heater systems is the safest and is able to control the temperature to a degree Celsius.

3. The base board 13 of this invention utilizes the northern ground heating system and the natural hole created aluminum frame work to serve as its delivery system. By sealing the ends of the aluminum and piping the hot water through newly prepared connectors, it becomes a natural heating method as well as a closed water loop. This allows the internal temperature to be regulated, which gives the cells a proper environment.

4. The anion distributor 3 is a crucial part in a cell's metabolic cycle by cleaning up any free radicals before new cells lose their existing anions to the dying free radicals.

5. The oxygen supplier 4 is to provide the bed 1 adequate oxygen, Cells need only one other item as sustenance other than temperature, and that is oxygen. Adequate oxygen levels is crucial to the creation of healthy cells.

6. The ultraviolet sanitation light 6, cleanliness is an important part of the sealed room, thus during the preheating period the light is activated to sterilize all corners.

While the preferred embodiments of the invention have been described above, it will be recognized and understood that various modifications may be made therein and the appended claims are intended to cover all such modifications that may fall within the spirit and scope of the invention.

What is claimed is:

1. An energizing bed comprising:
an enclosed space for a bed body, with two air holes, outward-opening doors on one side and a base board for the following devices;
a heating apparatus, installed under said base board of said bed, being the source of heat;
an anion generator, installed outside the unit of said bed, with pipes to funnel anions into the enclosed space;
an oxygen supplier, set the outside the unit of said bed, said oxygen supplier composed of the oxygen generator and its feed pipe, with the feed piping to deliver oxygen into the enclosed space;
a power control box, set on the outside the unit of said bed, said power control box connected to said heating apparatus, said anion generator, and said oxygen supplier to supply power and control power feed to said apparatus; and
Foresaid members composed together to make an enclosed space as a heated bed, supplying oxygen and anions to help a body accelerate cell recovery and become healthy.

2. The energizing bed as claimed in claim 1, wherein said bed has additional heating implements to quickly reach the desired temperature in the enclosed space.

3. The energizing bed as claimed in claim 1, wherein said bed located on top installed an ultraviolet light for keeping the enclosed space for sanitizing.

4. The energizing bed as claimed in claim 1, wherein said heating apparatus will be connected to a closed pump circulation system, allowing for said base board of said bed to be heated as water flows through them in a sealed circuit.

5. An energizing bed comprising:
an enclosed space for a bed body, with two air holes, outward-opening doors on one side and a base board for the following devices;
a heating apparatus, installed under said base board of said bed, being the source of heat;
an anion generator, installed outside the unit of said bed, with pipes to funnel anions into the enclosed space;
an oxygen supplier, set outside the unit of said bed, said oxygen supplier composed of the oxygen generator and its feed pipe, with the feed piping to deliver oxygen into the enclosed space;
a power control box, set on the outside the unit of said bed body, said power control box connected to said heating apparatus, said anion generator, and said oxygen supplier to supply power and control power feed to said apparatus; and
Foresaid members composed together to make an enclosed space as a heated bed, supplying oxygen and anions to help a body accelerate cell recovery and become healthy.
